(19) United States
(12) Patent Application Publication
(10) Pub. No.: US 2017/0089896 A1
(43) Pub. Date: Mar. 30, 2017

(54) ALLOGRAFT REJECTION TEST

(71) Applicant: MICHELE T. ROONEY, SPOKANE, WA (US)

(72) Inventor: MICHELE T. ROONEY, SPOKANE, WA (US)

(21) Appl. No.: 15/283,230

(22) Filed: Sep. 30, 2016

Related U.S. Application Data
(60) Provisional application No. 62/235,516, filed on Sep. 30, 2015.

(51) Int. Cl. G01N 33/569; C07K 16/18; A61K 51/10

(52) U.S. Cl. CPC G01N 33/56972; A61K 51/1096; C07K 16/18; G01N 2800/245; G01N 2800/56

(57) ABSTRACT

Systems and methods apply antibody labeling to quantify perforin-positive lymphocytes per unit area in an allograft biopsy, for detection and characterization of transplant rejection. An example technique includes staining the allograft biopsy to visualize lymphocytes, applying labeled anti-perforin antibodies to the allograft biopsy, visualizing the labeled anti-perforin antibodies to show perforin-positive lymphocytes, quantifying the perforin-positive lymphocytes to determine a count of the perforin-positive lymphocytes per unit area, and classifying a rejection parameter of the allograft based on the count. The example technique can be used to stratify patients into distinct risk groups with regard to presence of and type of allograft rejection. An example imaging system introduces a labeled anti-perforin antibody into a transplanted tissue in vivo, obtains images of the in vivo labeled anti-perforin antibody in the transplanted tissue, and determines a presence or a degree of tissue rejection based on the images.

### RELATED APPLICATIONS

This patent application claims the benefit of priority to U.S. Provisional Patent No. 62/235,516 to Rooney, filed Sep. 30, 2015, and incorporated herein by reference in its entirety.

### BACKGROUND

In medicine, an allograft is a tissue graft from a donor of the same species as the recipient, but not genetically identical. Recipients of transplanted organs (allografts such as kidney, heart, liver, lung, and pancreas transplants) are at risk of allograft rejection, which is an aggressive host immune response to the implanted organ that can damage and potentially destroy the transplanted organ. Transplant recipients receive drugs to suppress the immune system in order to make the organ or tissue rejection less likely. On the other hand, too much immune suppression can place the organ recipient at risk for severe complications, such as infection and malignancy. So, a balance must be pharmacologically maintained between the risk of organ rejection and the risk of severe complications.

Transplanted organs are most definitively monitored for evidence of immune rejection by way of tissue biopsy of the organ. For some transplanted organs, such as the heart, biopsies of the transplanted organ are routinely obtained as part of a rejection surveillance protocol. For other organ types, tissue biopsy is often obtained only when there is prior evidence of dysfunction according to a clinical monitor, such as a rising serum creatinine level, in the case of kidney transplant. In all cases, it is crucial to survival of the patient and the organ that a pathologist makes accurate and definitive assessments as to whether allograft rejection is present.

In monitoring for evidence of immune rejection of the allograft, the tissue biopsy is most often examined by at least two study methods, by light microscopy and by immunofluorescence. For kidney transplants, for example, a third modality, electron microscopy, can also be used.
For light microscopy, a portion of the biopsy is fixed in formalin to preserve the tissue. The tissue is permeated by and embedded in paraffin so that it can be cut on a microtome in thin (2-4 micron) intact sections. The sections are mounted on glass slides and stained with a series of chemical stains, and then examined under a standard light microscope by a pathologist for microscopic evidence of rejection.
For immunofluorescence microscopy, a non-fixed portion of the specimen is frozen within a rigid-when-cool cutting medium and cut in thin (4-5 micron) intact sections in a cryostat. The sections are mounted on glass slides. The tissue is then stained using fluorescein-labeled antibodies to inflammatory markers, such as complement factors C4d and C3d, and examined under a fluorescence microscope by a pathologist looking for patterns of expression of these markers that are associated with organ rejection.

Using both of these microscopy modalities, the trained pathologist looks for microscopic evidence of transplant rejection, as well as for any other processes that might also produce dysfunction in the transplanted organ. Current internationally recognized standard diagnostic criteria used by pathologists to diagnose kidney, liver and pancreas allograft rejection are delineated in the “Banff Classification for Allograft Rejection.” Current internationally recognized standard diagnostic criteria used by pathologists to diagnose heart and lung allograft rejection are delineated in the “International Society of Heart and Lung Transplantation” (ISHLT) schema.

For each organ system there are two recognized biological patterns of allograft rejection. One is primarily a cellular response by way of cytotoxic T lymphocytes that have become specifically activated against donor antigens and which then directly infiltrate, attack and injure the engrafted organ (T-cell mediated rejection, or TCMR). The other is a primarily a “humoral” response, wherein the organ recipient's immune system produces donor (allograft)-specific antibodies to the donor organ, leading to an immune assault upon and injury to the engrafted organ (antibody mediated rejection, or ABMR).

The diagnosis of transplant rejection can be quite difficult for the pathologist, for multiple reasons. ABMR is particularly difficult to identify in its earliest phase, which is the point where intervention is most effective. This is because antibodies cannot be seen directly under the microscope, and the pathologist must instead identify patterns of injury that antibodies can induce in order to indirectly determine that antibodies are there. These changes are often subtle, and there are several processes other than rejection that can produce changes quite similar to those seen in ABMR.

The processes that can mimic rejection, and that can fool the diagnosing pathologist, are particularly common in the transplant recipient population. For example, using transplanted kidney rejection as an example, some processes that can mimic ABMR in the transplant population include, among others: 1) dehydration, caused by diuretics and other medications; 2) infections, due to the immunosuppressive drugs that transplant recipients must take to prevent rejection; 3) direct kidney toxicity caused by the immunosuppressive agents; 4) recurrence in the transplanted kidney of the same renal disease that caused the native kidneys to fail in the first place; and 5) an unrelated de novo renal disease in the allograft. All of these processes can produce changes that are difficult to distinguish from bona fide transplant rejection, especially in the early stage of graft rejection, and multiple factors may be at play in any given case.

FIG. 1 shows a biopsy of kidney tissue in acute rejection with an accumulation of inflammatory cells in the capillary microvasculature. The known literature shows a strong association between accumulation of inflammatory cells in the microvasculature of the transplanted organ, termed “microvascular inflammation” or MVI, and the presence of rejection, both ABMR and TCMR. This association concept is best developed in the analysis of example kidney transplant rejection, but there is also good evidence that the relationship between MVI and rejection holds true in solid organ transplantation of all types. It is specifically the inflammatory cells that accumulate within the capillary vessels that have a strong relationship with acute rejection, and in the case of kidney rejection, not inflammatory cells that accumulate in the interstitium (the connective tissue between the cellular structures, such as renal tubules and the glomeruli) or other compartments of the tissue, which do not participate in this strong association with active rejection.

Conventional quantification of microvascular inflammation has indeed been incorporated into the diagnostic schema for kidney ABMR in the most recent (2013) update of the Banff Classification. In the kidney, the microvasculature includes both the capillaries that form the glomerular tufts, and the capillary network supplying the renal tubules (peritubular capillaries). The method currently used for quantification of microvascular inflammation in the Banff Classification requires the determination of a microvascular inflammation score (MVI), calculated as follows: MVI=g+ptc, where “g” is a score representing inflammatory cell infiltration of glomerular capillaries, and “ptc” is a score representing inflammatory cell infiltration of the peritubular capillaries. The glomerular score (g) requires the identification of occlusive intracapillary accumulations of inflammatory cells associated with evidence of capillary injury (such as endothelial cell swelling), as well as a determination of the percent of glomeruli so affected: resulting in a score of 1 for less than 25%, 2 for 25-75%, and a score of 3 for greater than 75% glomeruli affected.

The peritubular capillary score (ptc), on the other hand, requires visual assessment of the percent of peritubular capillaries containing inflammatory cells. Once a minimum scorable threshold of 10% of peritubular capillaries containing inflammatory cells for this value is observed, a score is then determined by counting the maximum number of inflammatory cells per vessel, yielding a score of 1 for less than 5 cells per peritubular capillary, 2 for 5-10 per capillary, and 3 if there are greater than 10 cells per peritubular capillary. The sum of g+ptc (MVI) is then applied in the Banff schema for allograft rejection. An MVI of 2 or greater is adequate to classify the biopsy as suspicious for ABMR.

The above MVI method, just described, for determining the existence or non-existence of allograft rejection is both vague and cumbersome, and particularly in early ABMR, when the MVI may be mild, inter-observer agreement has been quite poor (and so unreliable).

### BRIEF DESCRIPTION OF THE DRAWINGS

Certain embodiments of the disclosure will hereafter be described with reference to the accompanying drawings, wherein like reference numerals denote like elements. It should be understood, however, that the accompanying figures illustrate the various implementations described herein and are not meant to limit the scope of various technologies described herein.

FIG. 1 is a diagram of a biopsy of kidney tissue in acute rejection with an accumulation of inflammatory cells in the capillary microvasculature.

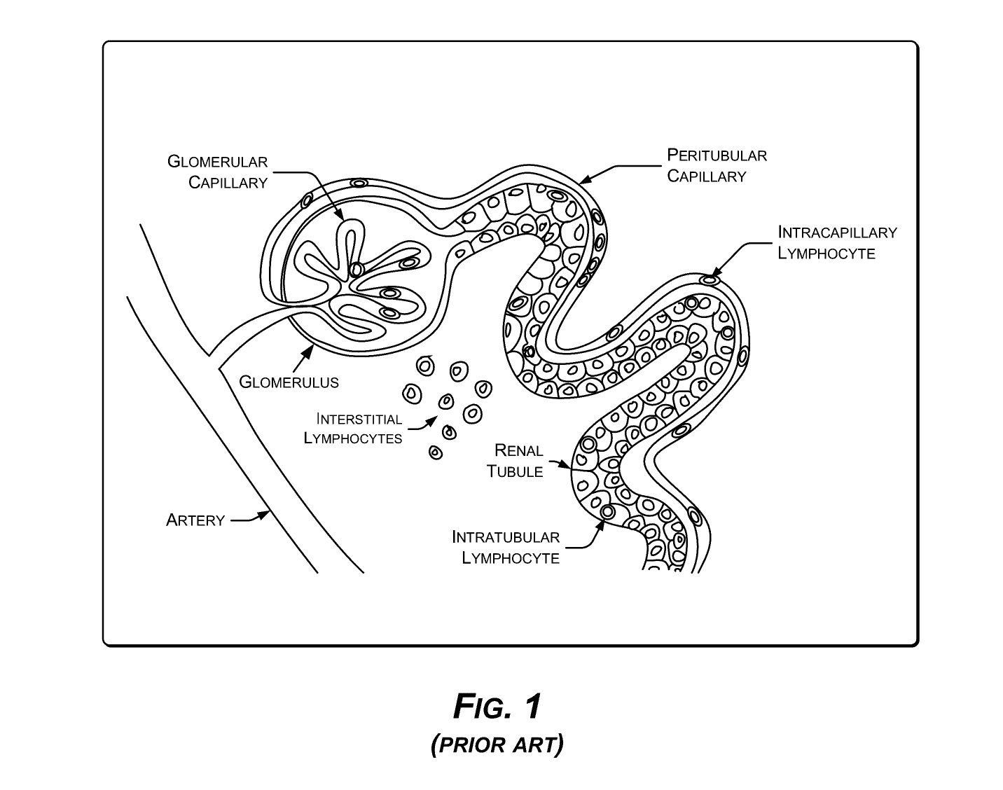

FIG. 2 is a diagram of visible changes that occur within microvascular inflammatory cells upon immunohistochemical staining for perforin.

FIG. 3 is a photograph of a kidney biopsy section with acute rejection as viewed in a microscope.

FIG. 4 is a flow diagram of an example method of determining presence of allograft (transplanted tissue) rejection.

FIG. 5 is a flow diagram of a more detailed method of determining a degree of allograft rejection.

FIG. 6 is a flow diagram of an even more detailed method of determining a degree of allograft rejection.

FIG. 7 is a flow diagram of an example method of applying antibody labeling to highlight perforin-positive lymphocytes in an allograft tissue biopsy, so that the perforin-positive lymphocytes can be readily quantitated per unit area.

FIG. 8 is a diagram of an example imaging method for determining allograft rejection in vivo.

FIG. 9 is an example table (Table (I)) with values for interpreting, characterizing, classifying, or staging the allograft rejection.

FIG. 10 is a flow diagram of an example process flow for classifying allograft (transplant) rejection, based on the cutoffs in Table (I) of FIG. 9.

FIG. 11 is a block diagram of an example hardware system for determining allograft rejection, based on perforin in lymphocytes involved in microvascular inflammation (MVI).

FIG. 12 is a diagram of an example imaging system for in vivo testing of allograft rejection.

### DETAILED DESCRIPTION

This disclosure describes improved test processes and systems for determining organ transplant (allograft) rejection. Example systems and processes apply antibody labeling to quantitate perforin-positive lymphocytes in an allograft biopsy per unit area, for detecting and characterizing (for example, classifying and staging) allograft rejection. Thus, in an implementation, a test for allograft rejection comprises immunohistochemical labeling of perforin-containing lymphocytes, and quantification of such perforin-containing lymphocyte cells in a transplant biopsy to gauge allograft rejection.

An example test procedure is developed from a new discovery that a high proportion of the intracapillary inflammatory cells that are present in the pathological context of allograft rejection are lymphocytes that contain the specific inflammatory mediator, perforin, which is detectable using a labeled anti-perforin antibody. Further, the example test procedure is based on a similar new discovery that very few such cells (referred to herein as anti-perforin detectable lymphocytes) are found outside of the microvasculature during transplant rejection. Thus, the example test procedure includes techniques to quantitate cells with anti-perforin antibody in a transplant biopsy, and is described herein for providing a clearer method for quantitating microvascular inflammation (MVI), thereby providing an improved test for allograft rejection.

### Example Processes

An example test process described herein quantifies the microvascular inflammation (MVI) characteristic of ABMR and TCMR based on a stain marker. Example systems and processes apply antibody labeling to quantitate perforin-positive lymphocytes in an allograft biopsy per unit area, for detection and characterization (staging, for example) of allograft rejection. Thus, in an implementation, an example test for renal allograft rejection comprises antibody labeling of perforin-containing lymphocytes, and quantification of such perforin-containing lymphocyte cells in a transplant biopsy to gauge allograft rejection.

Perforin is a pore forming cytolytic protein found in the cytotoxic granules of cytotoxic T lymphocytes (CTLs) and natural killer lymphocytes (NK cells). Also contained in these granules are serine proteases known as “granzymes.” When these cells kill a target cell, they do so by binding to the target cell, then forming a tight synapse, and then releasing the contents of these cytotoxic granules, including the perforin, into the synapse. The released perforin is in monomer form. Upon exposure to extracellular calcium ion during the process, the perforin monomer changes conformation making the outer surface more hydrophobic, facilitating intercalation of the monomer into the target cell membrane. Within the membrane of the target cell, perforin polymerizes and auto-assembles into pores, creating holes in the target cell membrane. The pores allow for passive diffusion of the granzymes, into the target cell which can cleave cellular proteins and also activate apoptosis (programmed cell death). Perforin is capable of lysing a variety of target cells. It is a key effector molecule for the CTL and NK cells.

Because perforin-stained cells stand out from the background, such cells can be easily quantitated per unit area either visually under a microscope by an operator, or by an image analyzer. In the case of example kidney rejection, compiled data confirm that this direct metric of quantitating perforin-stained microvascular inflammatory cells has the same associations with renal allograft rejection as the conventional microvascular inflammation (MVI) metric calculated as g+ptc, and provides improved efficacy, and also is a more direct and reproducible indicator for detecting allograft rejection. Moreover, an example perforin score described herein can effectively stratify patients into distinct risk groups, staging the patients with regard to the type or degree of allograft rejection with very little overlap in the distinct groups, strata, or stages. Thus, example systems and processes apply antibody labeling to quantitate perforin-positive lymphocytes in an allograft biopsy per unit area, for detection and characterization of allograft rejection.

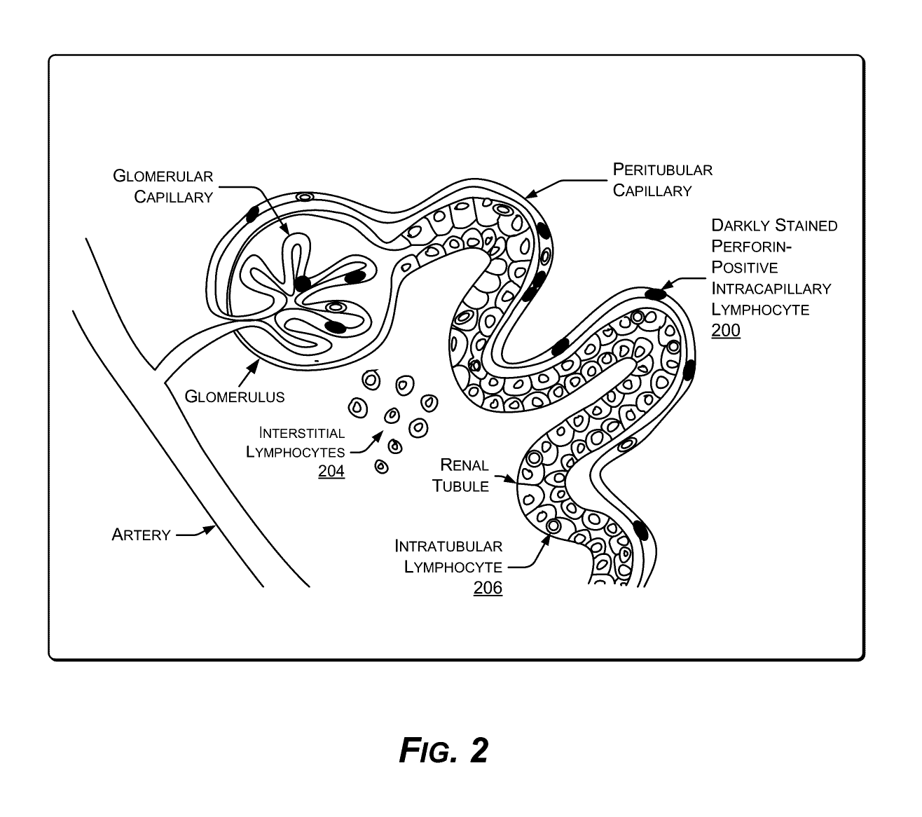

FIG. 2 illustrates visible changes that occur within microvascular inflammatory cells upon immunohistochemical staining for perforin. With light microscopy, the stained lymphocytes stand out as brown against a contrasting counterstain as observed by the human eye, or can be detected with an appropriately programmed image analyzer. These cells fluoresce yellow if stained with a fluorescent-labeled antibody and excited with a fluorescence microscope.

After immunohistochemical staining for perforin, the majority of intracapillary cells that are perforin-positive are darkly stained 200. Inflammatory cells of the interstitium 202 and intratubular lymphocytes 204 which are not part of the MVI remain largely unstained for perforin.

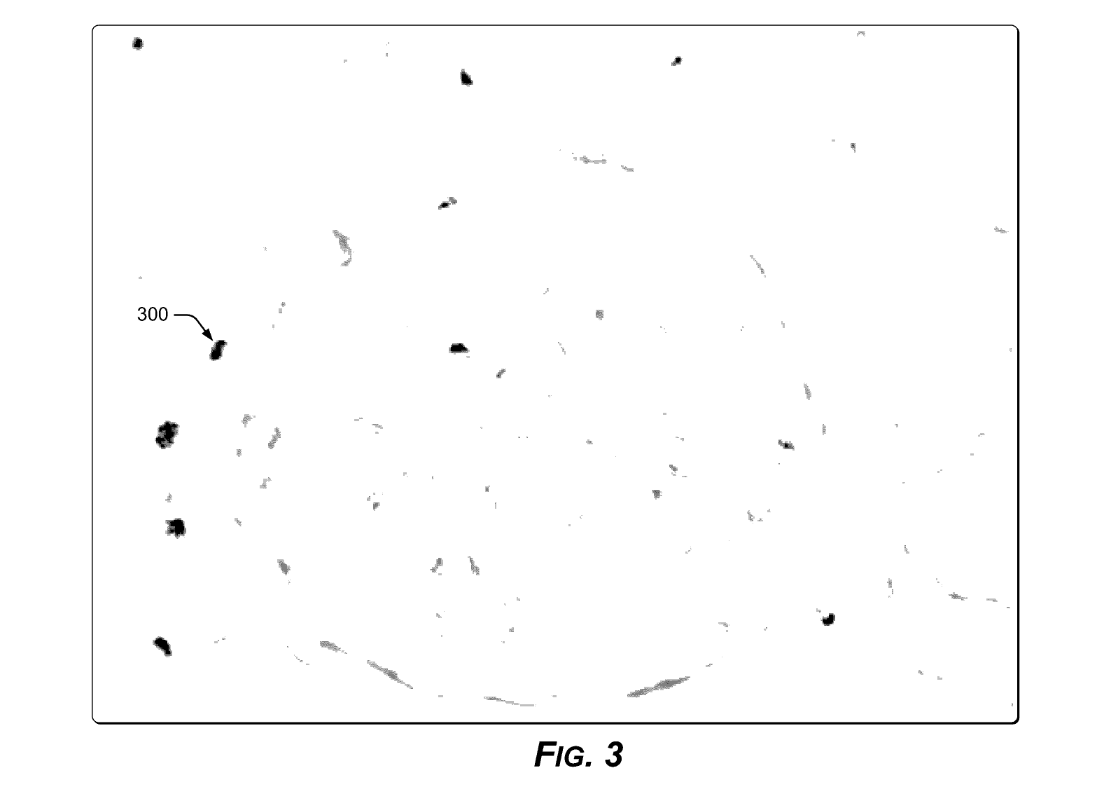

FIG. 3 shows a kidney biopsy section with acute rejection, as viewed in a microscope. In this black-and-white photomicrograph taken at 400×, perforin-containing granules within the cytotoxic T-lymphocytes in the capillary vessels appear darkly stained 300 against a pale background. The darkened cells 300 are located within the microvasculature. Lymphocytes that are visible in the interstitium (between the tubules and glomeruli) remain unstained.

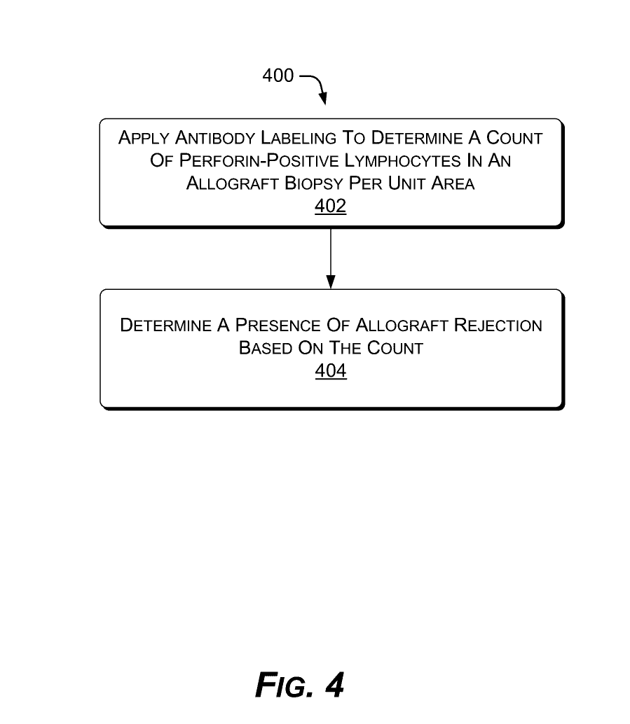

FIG. 4 shows an example method 400 of determining presence of allograft (transplanted tissue) rejection. In the flow diagram 400, operations are shown in individual blocks.

At block 402, antibody labeling is applied to the allograft biopsy to determine a count of perforin-positive lymphocytes per unit area.

At block 404, the presence and degree of allograft rejection, if any, is determined based on the count.

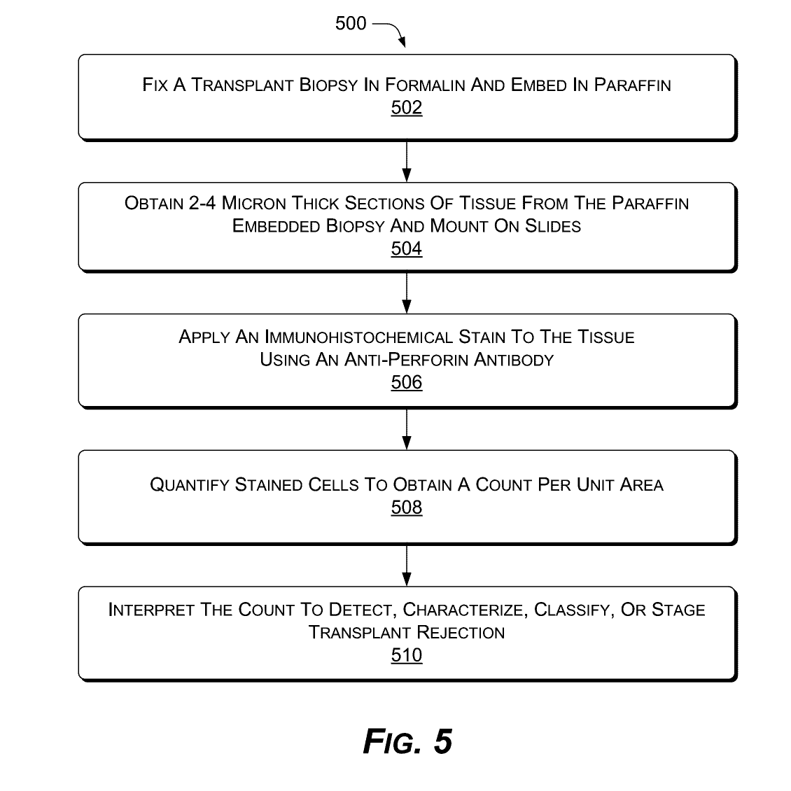

FIG. 5 shows a more detailed method 500 of determining a degree of allograft rejection. In the flow diagram 500, operations are shown in individual blocks.

At block 502, a transplant biopsy is fixed in formalin and embedded in paraffin.

At block 504, sections of tissue that are 2-4 microns thick are obtained from the paraffin-embedded biopsy and mounted on slides.

At block 506, an immunohistochemical stain is applied to the tissue using anti-perforin antibodies.

At block 508, stained cells are quantified to obtain a count of the stained cells per unit area.

At block 510, the count is interpreted, in order to detect, characterize, classify, or stage the allograft or transplant rejection.

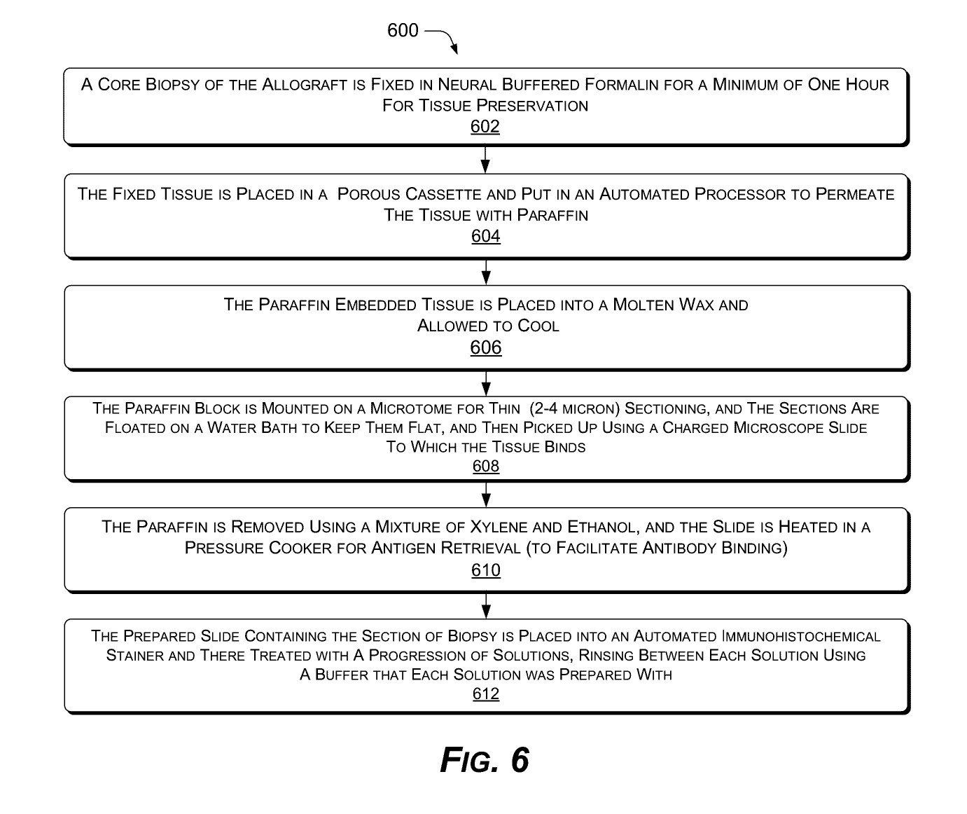

FIG. 6 shows an even more detailed method 600 of determining a degree of allograft rejection. In the flow diagram 600, operations are shown in individual blocks.

At block 602, the transplant biopsy is fixed in a solution of 10% neutral buffered formalin, for example, for a minimum of one hour, for tissue preservation.

At block 604, the biopsy tissue is placed in a porous container or cassette and then into an automated tissue processer, where the tissue is dehydrated in solutions of increasing ethanol concentration. The alcohol is cleared in xylene, and then the dehydrated tissue is permeated by molten paraffin wax.

At block 606, the paraffin infiltrated tissue is embedded in a block of paraffin and allowed to cool for sectioning/slicing with a microtome.

At block 608, the paraffin block is mounted on a microtome, and then 2-4 micron thick sections are sliced from the block and floated on a water bath to keep them flat, and then picked up by charged microscope slides to which the tissue binds, and allowed to dry.

At block 610, the tissue is deparaffinized in a mixture of xylene and ethanol, rehydrated, heated in a pressure cooker for antigen retrieval to facilitate antibody binding, and then rinsed in deionized water. The microscope slides may be treated with a peroxide blocking agent, for example for ten minutes, to block native peroxidase from creating artifact.

At block 612, the prepared slides, each containing a section of the biopsy are placed into an automated immunohistochemical stainer and treated or incubated with a progression of solutions, rinsing between each solution using a buffer that each solution was prepared with.

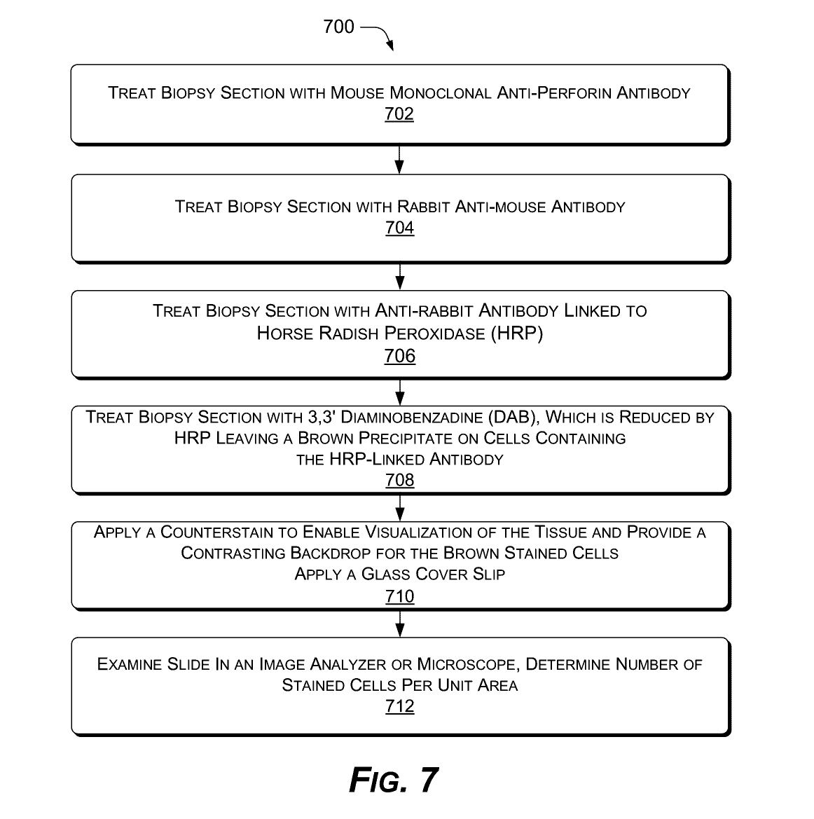

FIG. 7 shows an example method 700 of applying antibody labeling to highlight perforin-positive lymphocytes in an allograft tissue biopsy, so that they can be readily quantitated per unit area. In the flow diagram 700, operations are shown in individual blocks. In this example method 700, a chromogen-labeled anti-perforin antibody is applied to paraffin-embedded fixed tissue. It should be noted that perforin-containing lymphocytes could alternatively be highlighted on cryosection of the biopsy using fluorochrome-labeled antibody, and then examined on a fluorescence microscope for quantitation.

At block 702, the biopsy section is treated with mouse monoclonal anti-perforin antibody, for example, for 30 minutes.

At block 704, the biopsy section is treated with rabbit anti-mouse antibody, for example, for 10 minutes.

At block 706, the biopsy section is treated with anti-rabbit antibody linked to horse-radish peroxidase (HRP), for example, for 10 minutes.

At block 708, the biopsy section is treated with chromogenic detection compound 3,3′ diaminobenzidine (DAB) (which undergoes oxidization by the HRP, leaving a brown precipitate on the cells containing perforin), for example, for 10 minutes.

At block 710, a counterstain, such as a (contrasting) hematoxylin stain, is selected to enable visualization of the tissue, and applied to the biopsy section. Dehydration is then performed and a cover slip can be applied.

At block 712, quantification can be performed by a standard image analyzer. The slides are placed on the image analyzer, programmed to detect the brown color of the DAB chromogen, and to count the number of “events” (brown cells) per unit area. Alternatively, a count of labeled cells per ten (10) high power microscope fields can be recorded by an operator.

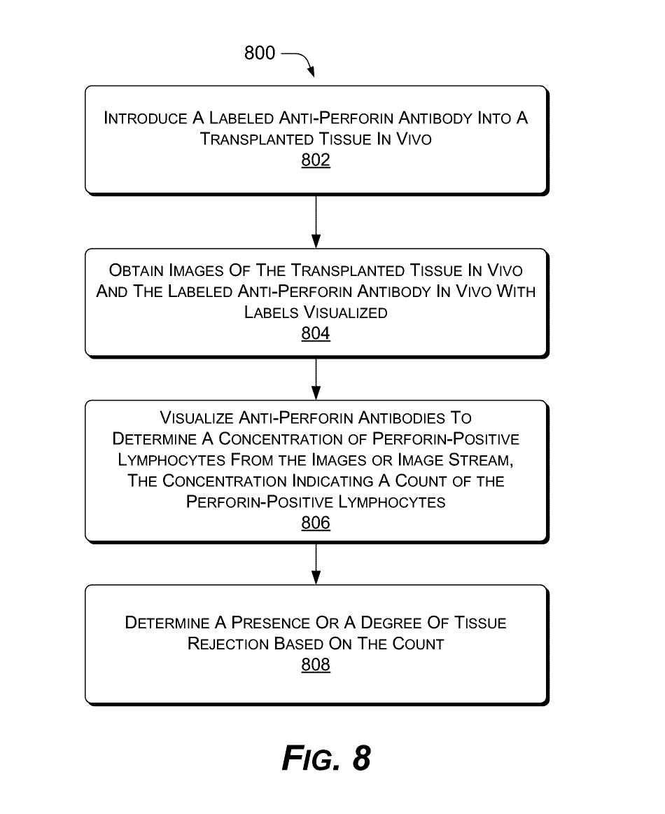

FIG. 8 shows an example imaging method 800 for in vivo determining allograft rejection. In the flow diagram 800, operations are shown in individual blocks.

At block 802, labeled anti-perforin antibodies are introduced into a transplanted tissue, in vivo, that is, into a patient.

At block 804, images or an image stream of the transplanted tissue in vivo are obtained, including the labeled anti-perforin antibodies, with labels visualized in vivo.

At block 806, the labeled anti-perforin antibodies are visualized to determine a concentration per unit area or per unit volume of perforin-positive lymphocytes from the one or more images or the image stream, the concentration indicating a count of the perforin-positive lymphocytes.

At block 808, a presence of a degree of tissue rejection is determined, based on the count.

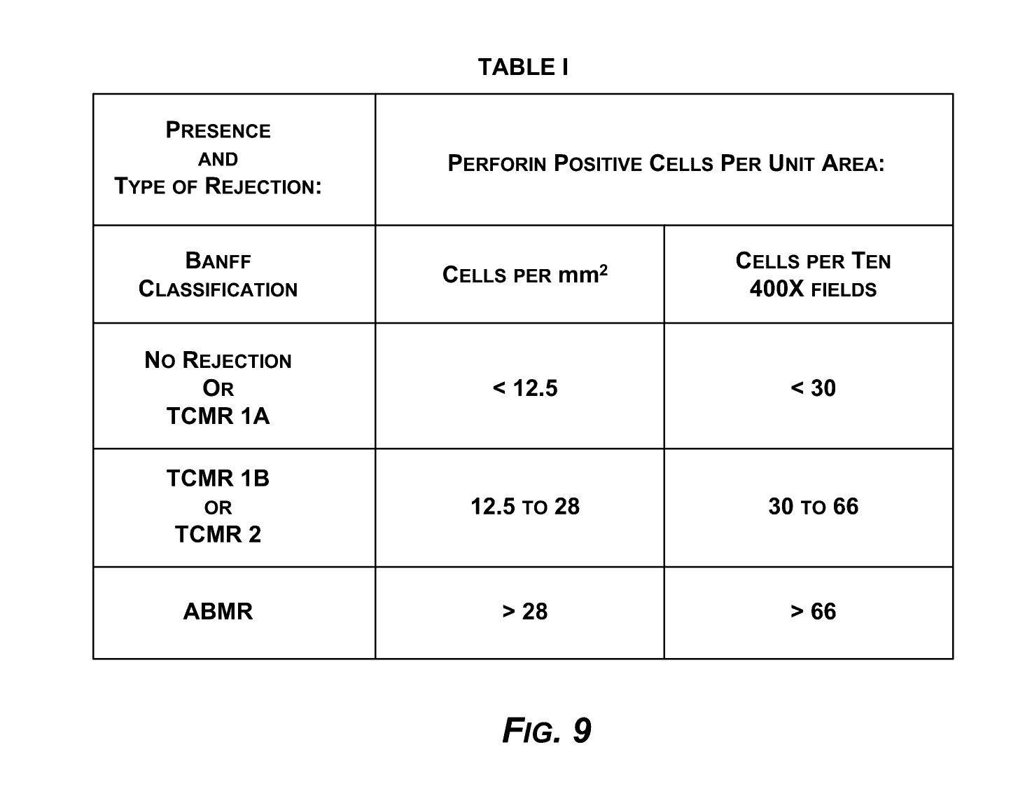

FIG. 9 is an example table (Table (I)) for interpreting, characterizing, classifying, or staging the allograft rejection. The count value, comprising perforin-positive cells per unit area, is interpreted using cutoffs determined by accumulated patient data, exemplified by example Table 1 in FIG. 9. The cutoff or threshold values shown in Table (I) may be determined by quantitating perforin-positive cells in transplant biopsies received over a twelve month period, for example. The biopsies are categorized as to the presence and type of rejection by trained pathologists using the current Banff criteria (as an example, for renal rejections). As with all laboratory tests, the specific cutoffs or thresholds can be validated and authenticated for the testing laboratory and its unique equipment (in order to certify the testing laboratory for performing the test).

In the example table, Table (I), no statistically significant difference in perforin count was demonstrated between kidney biopsies with mild cell mediated rejection (CMR 1A) and non-rejected kidneys.

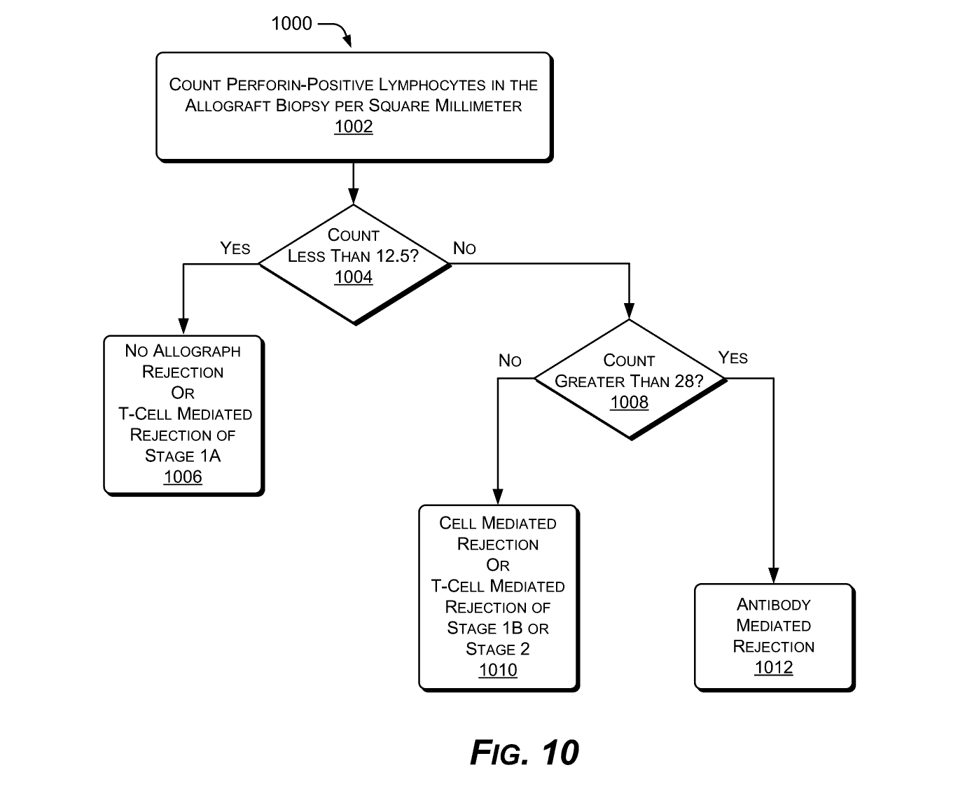

FIG. 10 shows a flow diagram for classifying allograft (transplant) rejection, based on the cutoffs in Table (I) of FIG. 9. In the flow diagram 1000, operations are shown in individual blocks.

At block 1002, a count is obtained, of perforin-positive lymphocytes in the allograft biopsy, for example, per square millimeter.

At block 1004, the count is evaluated to determine if the count is less than or equal to 12.5 per square millimeter.

If the count is 12.5 or fewer, then at block 1006, there is no allograft rejection, or the allograft rejection is a T-cell mediated rejection of Stage 1A.

If the count is greater than 12.5, then at block 1008 the count is evaluated to determine if the count is greater than 28. If the count is not greater than 28, then at block 1010, the allograft rejection is a cell mediated rejection, or a T-cell mediated rejection of Stage 1B or Stage 2.

If the count is greater than 28, then the allograft rejection is antibody mediated rejection.

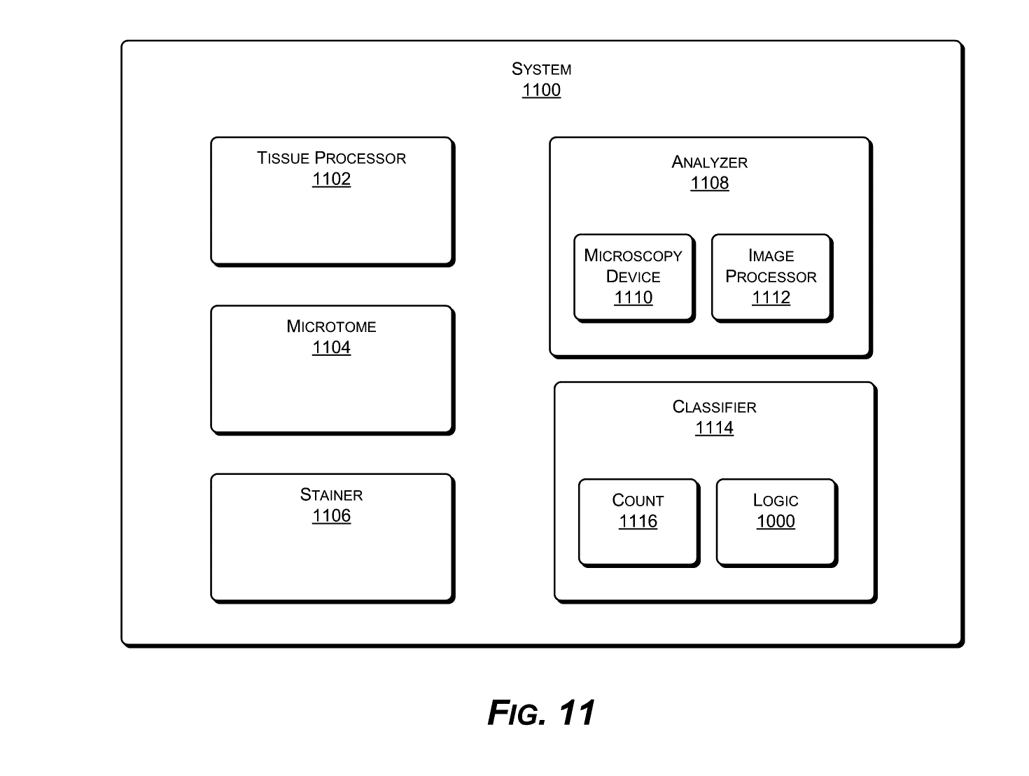

FIG. 11 shows an example hardware system 1100 for determining allograft rejection, based on perforin in lymphocytes involved in microvascular inflammation (MVI). The example system 1100 includes a tissue processor 1102, a microtome 1104, a stainer 1106, an analyzer 1108, including a microscopy device 1110 and an image processor 1112. The example system 1100 also includes a classifier 1114, including a tangible data storage medium, such as a computer memory or storage drive, to store the count 1116, and includes the interpretation/classification logic 1000 as shown in FIGS. 10-11, for example.

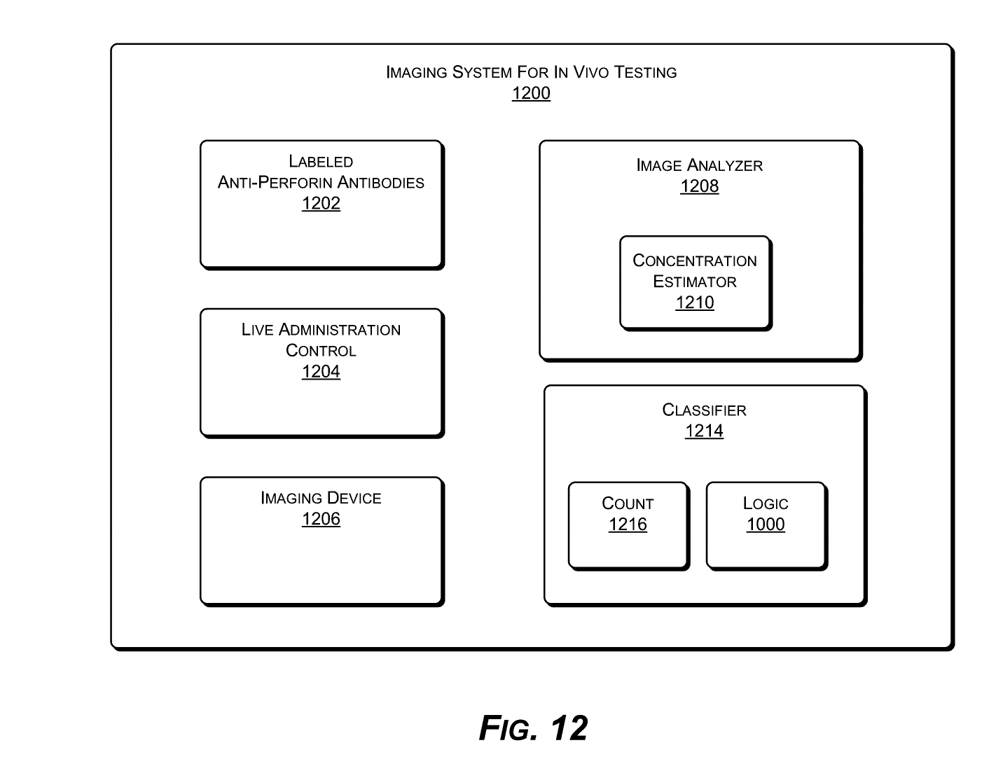

FIG. 12 shows an imaging system 1200 for in vivo testing. The example imaging system 1200 includes production, storage, and handling hardware for labeled anti-perforin antibodies 1202, such as radio-labeled antibodies; live administration control 1204 for real-time administration of labeled anti-perforin antibodies 1202 to a patient, and an imaging device 1206, which can rely on numerous sound, light, electromagnetic radiation (e.g., x-ray), and magnetic resonance techniques available in radiology to obtain images or an image stream. An image analyzer 1208 visualizes the labeled anti-perforin antibodies 1202 in the images or image stream, and may include a concentration estimator 1210, which measures the perforin-positive lymphocytes in a given area or volume, and relates the concentration to a count 1216. A classifier 1214 interprets and classifies the count, according to the logic 1000 shown in FIGS. 10-11.

While the present disclosure has been done with respect to a limited number of embodiments, those skilled in the art, having the benefit of this disclosure, will appreciate numerous modifications and variations therefrom. It is intended that the appended claims may cover such modifications and variations as fall within the true spirit and scope of the disclosure.

1. A method of testing for allograft rejection, comprising:
applying antibody labeling to determine a count of perforin-positive lymphocytes in an allograft biopsy per unit area; and
determining a presence of allograft rejection based on the count.

2. The method of claim 1, further comprising determining a count of the perforin-positive lymphocytes in an intracapillary microvasculature of the allograft biopsy to determine a microvascular inflammation (MVI) score relating to the allograft rejection.

3. The method of claim 1, further comprising determining a degree of the allograft rejection based on the count.

4. The method of claim 3, wherein determining the degree of the allograft rejection comprises determining a presence of, or a degree of, a cell mediated rejection or an antibody mediated rejection based on the count.

5. The method of claim 1, further comprising classifying the allograft rejection based on the count of perforin-positive lymphocytes in the allograft biopsy per unit area, wherein the classifying further comprises determining a presence of, or a type of, the allograft rejection;
wherein a count of less than 12.5 perforin-positive lymphocytes per square millimeter indicates either no allograft rejection or a T-cell mediated rejection (TCMR) of stage 1A;
wherein a count of 12.6-30.0 perforin-positive lymphocytes per square millimeter indicates either a cell mediated rejection, a TCMR of stage 1B or stage 2; and
wherein a count of more than 30.0 perforin-positive lymphocytes per square millimeter indicates an antibody mediated rejection (ABMR).

6. The method of claim 1, wherein classifying the allograft rejection based on the count of perforin-positive lymphocytes in an allograft biopsy per unit area further comprises determining a presence of, or a type of, the allograft rejection;
wherein a count of less than 30 perforin-positive lymphocytes per ten 400× power visual fields indicates no rejection or a 1A stage;
wherein a count of 31-66 perforin-positive lymphocytes per ten 400× power visual fields indicates a cell mediated rejection, a 1B stage, or a 2 stage; and
wherein a count of more than 66 perforin-positive lymphocytes per ten 400× power visual fields indicates an antibody mediated rejection.

7. The method of claim 1, wherein the antibody labeling comprises an immunohistochemical or an immunofluorescent staining process.

8. The method of claim 1, further comprising:
fixing a tissue of the allograft biopsy in formalin and embedding the tissue in paraffin;
mounting 2-3 micron thick sections of the tissue from the paraffin onto microscope slides;
applying an immunohistochemical stain to the tissue using an anti-perforin antibody;
applying a contrast stain to the tissue when needed for visualization;
quantitating stained cells per unit area to obtain the count; and
classifying a rejection parameter related to the allograft based on the count.

9. The method of claim 1, wherein the allograft biopsy comprises a renal tissue.

10. The method of claim 1, further comprising:
staining the allograft biopsy to visualize lymphocytes;
applying labeled anti-perforin antibodies to the allograft biopsy;
visualizing the labeled anti-perforin antibodies to show perforin-positive lymphocytes;
quantifying the perforin-positive lymphocytes in the allograft biopsy to determine a number of the perforin-positive lymphocytes per unit area; and
determining a presence of allograft rejection based on the number of the perforin-positive lymphocytes per unit area.

11. The method of claim 1, wherein applying antibody labeling further comprises incubating a tissue of the allograft biopsy with a sequence of solutions, comprising:
a mouse monoclonal anti-perforin antibody solution;
a rabbit anti-mouse antibody solution;
an anti-rabbit antibody linked to horse-radish peroxidase (HRP);
a chromogenic detection compound of 3,3′ diaminobenzidine (DAB) to leave a brown precipitate on cells of the tissue containing perforin; and
a counterstain comprising a contrasting hematoxylin stain.

12. The method of claim 11, further comprising quantifying a microvascular inflammation (MVI) characteristic of an antibody mediated rejection (ABMR) and or a T-cell mediated rejection (TCMR) based on a stain marker.

13. A system, comprising:
a tissue processor for embedding a tissue of an allograft biopsy in a paraffin block;
a microtome for obtaining slices of the tissue; at least a slide for mounting the slices;
a stainer for applying an immunohistochemical stain to the tissue using an anti-perforin antibody, and for applying a counterstain when needed for visualization;
an analyzer to determine a count of stained cells per unit area; and
a classifier to determine a rejection status of the allograft biopsy based on the count.

14. The system of claim 13, wherein the analyzer comprises a light microscopy device or an immunofluorescence microscopy device.

15. The system of claim 12, further comprising threshold logic applied by the classifier;
wherein a count of less than 12.5 perforin-positive lymphocytes per square millimeter indicates no rejection or a 1A stage;
wherein a count of 12.6-30.0 perforin-positive lymphocytes per square millimeter indicates a cell mediated rejection, a 1B stage, or a 2 stage; and
wherein a count of more than 30.0 perforin-positive lymphocytes per square millimeter indicates an antibody mediated rejection; and
further comprising a user interface hardware to display the rejection status of the allograft biopsy.

16. An imaging method, comprising:
introducing a labeled anti-perforin antibody into a transplanted tissue in vivo;
obtaining one or more images or an image stream of the transplanted tissue in vivo and the labeled anti-perforin antibody in vivo;
visualizing the anti-perforin antibody to determine a concentration per unit area or per unit volume of perforin-positive lymphocytes from the one or more images or the image stream, the concentration indicating a count of the perforin-positive lymphocytes; and
determining a presence or a degree of tissue rejection based on the count.

17. The imaging method of claim 16, further comprising determining a degree of the tissue rejection based on the count.

18. The imaging method of claim 16, further comprising classifying the tissue rejection based on the count, wherein the classifying further comprises determining a presence of, or a type of, the tissue rejection;
wherein a count of less than 12.5 perforin-positive lymphocytes per square millimeter indicates either no tissue rejection or a T-cell mediated rejection (TCMR) of stage 1A;
wherein a count of 12.6-30.0 perforin-positive lymphocytes per square millimeter indicates either a cell mediated rejection, a TCMR of stage 1B or stage 2; and
wherein a count of more than 30.0 perforin-positive lymphocytes per square millimeter indicates an antibody mediated rejection (ABMR).

19. The imaging method of claim 16, wherein the labeled anti-perforin antibody comprises a radio-labeled anti-perforin antibody, and obtaining the one or more images of the transplanted tissue and the in vivo radio-labeled anti-perforin antibody further comprises obtaining the one or more images via a radiologic imaging technique.

20. The imaging method of claim 16, wherein an imaging hardware determines the concentration of the visualized labeled anti-perforin antibody per unit area or per unit volume indicating the count of the perforin-positive lymphocytes.
